(19) United States
(12) Patent Application Publication
(10) Pub. No.: US 2026/0054098 A1
(43) Pub. Date: Feb. 26, 2026

(54) ANATOMICALLY DRIVEN TRANSCRANIAL ULTRASOUND TREATMENT SYSTEM AND METHOD

(71) Applicant: Sanmai Technologies, PBC, Sunnyvale, CA (US)

(72) Inventors: Tomokazu SATO, Minneapolis, MN (US); Christopher DAFT, Tucson, AZ (US); Sarp SATIR, Oakland, CA (US)

(21) Appl. No.: 19/029,125

(22) Filed: Jan. 17, 2025

Related U.S. Application Data
(60) Provisional application No. 63/685,512, filed on Aug. 21, 2024.

(51) Int. Cl. A61N 7/00

(52) U.S. Cl. CPC A61N 7/00; A61N 2007/0026

(57) ABSTRACT

Systems and methods use the anatomy of the target brain region to suggest waveforms for optimal stretching of the target tissue for neuromodulation. Cell orientation, white matter tracts, or direction of connection between brain regions of the target region are considered. The timing of waveforms generated is based on time constants relevant to the target brain region and structures. The System can lock into the phase and frequency of the target using physiological measurements provided by EEG or other physiological signals. The system provides recommendations on the number of transducers to use, the placement of the transducers, and other ultrasound stimulation parameters. In an embodiment, the system generates waveforms where the spatial derivates of pressure are maximized to improve the efficacy of the stimulation.

### CROSS-REFERENCE TO RELATED APPLICATIONS

This application claims priority benefit of U.S. Provisional Application No. 63/685,512, entitled ANATOMICALLY DRIVEN TRANSCRANIAL ULTRASOUND TREATMENT SYSTEM AND METHOD, and filed Aug. 21, 2024. The subject matter of this related application is hereby incorporated herein by reference.

### BACKGROUND

### Field of the Various Embodiments

This application deals with transcranial ultrasound (TUS) waveforms and fields based on the target anatomy.

### SUMMARY

Transcranial ultrasound (TUS) systems use ultrasound (US) to modulate neural activity within the brain through the skull without surgery or other invasive methods. Current transcranial ultrasound systems aim to produce a focus or pressure maximum at the desired treatment location, similar to diagnostic ultrasound imaging systems. In particular, the system's goal is to maintain the tightest focus as measured by pressure or intensity. This approach of generating maximum pressure at the target brain structure with the tightest focus is far from optimal. While it has been shown to work, it is inefficient because it requires a higher than necessary ultrasonic intensity to achieve therapeutic goals. The efficacy shown in clinical trials is likely a side-effect of creating a tight focus.

For neuromodulation, the ultrasound field must be capable of stretching the target structures. The stretching of neurons is thought to open ion channels and lead to neural activity changes. Thus, the ultrasonic fields should focus on ultrasound parameters that maximize stretching rather than pressure. For example, generating a US force that moves the target brain tissue towards other fixed parts of the brain (the “anchor”) only creates slack in tissue and no stretching. Thus, the direction of the ultrasound field can play a critical role in effective stretching and ensuring effective modulation.

Studies have shown that modifying timing parameters like PRF (Pulse-Repetition Frequency) is necessary for efficacious and reliable results. Generally, the timing parameters are modified in a trial-and-error manner, which is time-consuming and counterproductive, especially when considering a patient's or subject's limited time commitments. There is a need for a better approach to ultrasound's timing parameters and other parameters. The ultrasound fields' shape and direction, waveform timing, and other parameters must be anatomically aware to maximize stretching and, thus, the neuromodulation.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

The figures are not geometrically or anatomically accurate and are only for illustration.

FIGS. 1A and 1B show an example implementation of a transcranial ultrasound system 100.

FIG. 2 shows example waveforms generated by System 100.

FIGS. 3A and 3B show the effects of US waveforms with a low and high F-number, respectively.

FIGS. 4A and 4B show examples of System 100 creating stretching along an axon or white matter (WM) tract.

FIG. 5 shows an example of System 100 stretching of a cell body attached to a well-anchored dendritic tree.

FIG. 6 shows an example of System 100 generating a field that generates orthogonal stretching of an axon.

FIG. 7A shows an example of System 100 using antiparallel, spatially limited standing waves for modulation.

FIG. 7B shows simulation graphs of antiparallel spatially limited standing waves.

FIGS. 8A and 8B show examples of System 100 generating antiparallel stretching.

FIG. 9A-9E show examples of System 100 creating stretching and strain within a single brain region.

FIG. 10 shows an example of System 100 targeting tracts from or to deeper brain regions.

FIG. 11 shows an example of System 100 collinear targeting of two regions.

FIG. 12 shows an example of method 1200 using System 100 for transcranial stimulation.

FIG. 13A-E shows example simulation results using apodize to increase gradient metrics.

### DETAILED DESCRIPTION

In the following description, numerous specific details are set forth to provide a more thorough understanding of the various embodiments. However, it will be apparent to one of skilled in the art that the inventive concepts may be practiced without one or more of these specific details.

A system and method use the anatomy of the target brain region to suggest waveforms for optimal stretching of the target tissue, whether cell bodies in a brain region or axons and white matter tracts. Cell orientation, white matter tracts, or direction of connection between brain regions of the target region are considered. The timing of waveforms generated is based on time constants relevant to the target brain region and structures. The System can lock onto the phase and frequency of the target using physiological measurements provided by EEG, or other physiological measurements. Such phase measurements may also be obtained or estimated indirectly. For example, they may be estimated from connected regions whose signals can be more strongly measured; through interpolation or other methods across brain regions; or by assessing excitability of the target region in time by sending excitatory ultrasound fields to the target brain structures and measuring the magnitude or phase delays of evoked potentials in EEG. The system provides recommendations on the number of transducers to use, the placement of the transducers, and other ultrasound stimulation parameters. In an embodiment, the system generates waveforms where the spatial derivatives of pressure are maximized to improve the efficacy of the stimulation. Waveform herein refers to the time development of the ultrasound supplied to the head, and “field” to refer to its spatial extent and pattern. This application discloses systems and methods of a system to inform and present a menu of efficacious waveforms derived from the known anatomical targets, individualized placements and options, and neurophysiology, which create the desired neural changes with lower intensity. This is useful because it reduces unwanted effects away from the target tissue, improves safety and lengthens battery life in portable devices.

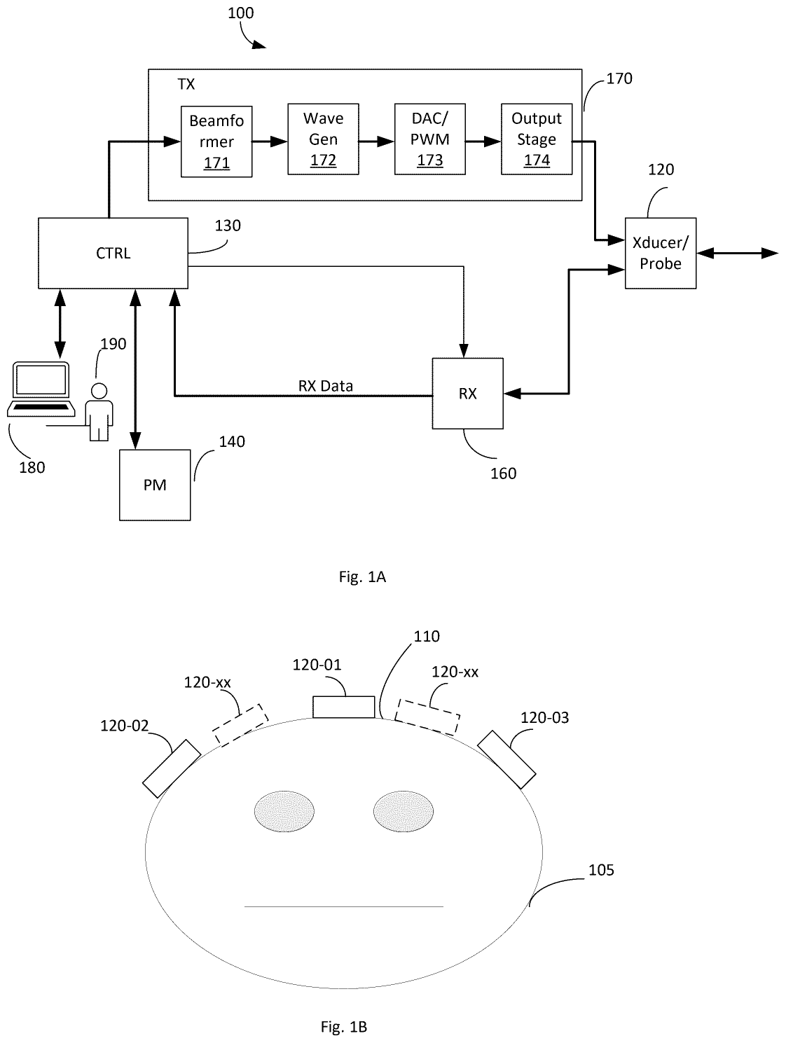

FIGS. 1A and 1B show an example implementation of a transcranial ultrasound system 100. FIG. 1A is an example block diagram of the system and FIG. 1B shows an example usage of System 100. In this system, the subject or patient 105 has one or more ultrasound transducers 120 placed on his head 110. TUS system 100 includes physiological measurement systems 140, such as EEG (electroencephalography), MEG (magnetoencephalography), EMG (electromyography), to monitor the efficacy of the stimulation. In an embodiment, System 100 can lock into the frequency and phase detected by physiological measurement (PM) system 140 and generate waveforms with the same frequency or phase. For example, the frequency and phase detected by the physiological measurement system 140 are the timing of patient 105's neuronal activity.

In an embodiment, System 100 can include one or more than one transducer. The figure labels the three transducers as 120-01, 120-02, and 120-03 and can include additional transducers shown as 120-xx. Transducer 120 can be a single-element transducer or an array transducer. The transducers 120 can be used for stimulation, guidance, or both. Neuromodulation or stimulation can noninvasively modulate neural activity. In an embodiment, the real-time imaging data from the transducer 120-xx controls the timing of waveforms generated by System 100. Control 130 controls System 100. Control 130 includes one or more processors (such as CPUs, AI processors, and GPUs), storage, and memory (volatile and non-volatile). A clinician 190, can interact with System 100 via UI 180. UI 180 includes displays, keyboard, mice, etc., for interaction between System 100 and the clinician. UI 180 can display physiological data from EEG/EMG 140 or RX imaging Data from transducer 120.

System 100 displays UI elements such as a menu or graphical images of the brain that allow clinician 190 to select one or more target areas. System 100 provides a suggested placement (position and pose) for probe 120 based on the selected target areas. System 100 also suggests the number of transducers/probes 120 and the ultrasound parameters based on the target locations selected. The parameters include focal depth, pressure gradient, pressure, and the timing of the waveforms generated. System 100 guides placing transducer 120's location based on real-time data gathered from Subject 105. Real-time data is from one or more of the following: neuronavigational systems (not shown in the figure), physiological measurements 140, or imaging performed by transducers 120. System 100 may use other data, such as MRI (magnetic resonance imaging) or CT-scan (computed tomography), to guide the placement of probe 120. System 100 considers subject 105's unique physical characteristics, such as hair, thick bone structure, etc., when providing probe 120 placement guidance. In addition, System 100 may consider preferences such as not delivering the ultrasound through hair if alternative solutions exist.

Even though Clinician 190 may have selected a single target region, System 100 may suggest applying the ultrasound field to one or more target regions. For instance, System 100 may suggest aiming at two sides of the target region alternately, causing cyclic stretching between the left and right sides of the target. This can be done using a single transducer 120 or more than one transducer 120.

System 100 provides aid to help clinician 190 visualize the fields, the interaction of one or more fields superimposed on the target, the stretching caused, etc. For example, the results of a simulation are displayed to clinician 190 on UI 180. System 100 uses Patient 105's MRI data (MRI performed beforehand) or generic brain data for performing the simulation. Real-time data (e.g., from RX imaging using transducer 120) can also be used. Clinician 190 can modify the parameters such as focus, any correction parameters, ultrasound pressure gradient or pressure, direction, etc. Real-time data such as EEG (physiological measurement 140), RX imaging data from transducer 120, evoked potentials, etc., can help aid System 100 and Clinician 190 in modifying the ultrasound parameters to improve the stimulation efficacy. System 100 and Control 130 also will simulate ultrasound field transformation and aberration due to the skull and perform phase corrections. As an example, suppose the clinician wishes to create a high NA (numerical aperture) field to create maximal tissue stretching within the brain. Simply using the array element parameters to generate such fields in water would not lead to optimal field generation beyond the skull. As the locations of maximal stretching depend highly on the field, the skull is known to aberrate the field shape, and the aberrations from a skull highly individualized to each subject, these will need to be corrected considering the individual's anatomy. The skull can often cause lateral deflections due to refraction, as well as shortening the focus and acting as a lens. Both effects can lead to missing the intended target. System 100 uses high-quality simulations allowing for accurate positioning and proper shape of the ultrasound field in the subject's skull to achieve effective neuromodulation.

RX block 160 in System 100 handles receive functions and includes electronics such as AFE (analog front end), beamformer, signal processing blocks, etc., for imaging Subject 105's brain. RX block 160 can also be used to receive real-time data to improve the efficacy of the stimulation. For example, in FIG. 1B, transducer 120-01 is used for stimulation only, while transducers 120-02 and 120-03 are used for real-time imaging only. Other configurations are possible, where the functionality of transducers changes in real-time.

TX block 170 in System 100 handles the transmit functions. TX block 170 includes functions such as a beamformer 171, wave generator 172 (for generating waveforms), DAC/PWM 173 (Digital to Analog, Pulse width modulation—for digital to analog conversion), output stage 174 (for driving voltage amplitudes to the transducer 120 elements), etc. TX block in System 100 generates waveforms with timing based on time constants relevant to target brain regions. The target brain region(s) is gathered from clinician 190's input. The timing of waveforms is further refined by real-time data, where the frequency and phase of the waveforms are further adjusted to match the timing of the neuronal activity of the target brain regions. In an embodiment, System 100 generates waveforms that optimize the ultrasound field shape, considering the target brain's anatomy, such as cell orientation, white matter tracts, the direction of connection between brain regions, etc. For example, the pressure field generated creates stretching orthogonal to a white matter tract, axons, etc. In a different example, a moving pressure field with a short focus traverses along the length of an axon, WM (white matter) tract, etc. In another example, traveling waves generated by two or more transducers are used. The transducers are antiparallel and operate at different frequencies to eliminate standing waves. A shifting frequency or a chirp waveform is used in a different example. In a less preferred example, standing waves are generated. Standing waves can reduce tight spatial focus (in the form of repeating patterns of nodes and antinodes whose spatial extent can last over multiple wavelengths, especially if the focal zone length is long) and may cause unpredictable high forces or off-target neuromodulation. Standing waves can be generated by opposing transducers or from reflections from the skull that are distal to the transducer.

In an embodiment, System 100 optimizes the pressure spatial derivative or gradient, as US fields with large gradients give more efficacious neuromodulation. System 100 maximizes the ratio of the spatial pressure gradient to the maximum pressure by optimizing the apodization function in the output state 174 to optimize the waveform generated.

Control 130 is a single block in this implementation but can be implemented as a distributed system where different electronics (TX 170 and RX 160 functions) and cabling control each transducer 120 individually. Control 130 controls the timing and phase of the ultrasound waveforms generated by the transducers 120. For example, transducers 120-01 and 120-02 have the same frequency, but transducer 120-02 can have a phase offset from transducer 120-01, while transducer 120-03 has a different frequency. Having more than one transducer 120 can be advantageous and can help provide a more effective treatment. More transducers 120 can help compensate for skull curvature by allowing for better contact between the transducer and the head. This allows for a larger aperture to be used, which can be necessary for more efficacious ultrasound fields. More transducers 120 allows for coverage for a wide variety of anatomy. This is especially helpful when one or more transducers' field is occluded in their path to the target. The path to the target may be blocked due to skull bone or any blood vessel one may want to avoid. Two or more transducers 120 generate stretching of a target area by pulling the target area apart using two (or more) transducers where the ultrasound fields are focused at regions beyond the target area. In a similar embodiment, two (or more) transducers pull apart different connected brain regions and stretch the axonal connections between the two regions. More transducers 120 can achieve better modulation while stretching the two regions. Careful consideration of the target anatomy, direction of the desired axon or brain region movement, noise, and any path obstacles are required to decide the number of transducers 120.

### Ultrasound Timing

Neuronal activity has a natural frequency. For example, Theta waves have a frequency range of 4-8 Hz and are associated with the oscillation used by the hippocampus to connect to other brain circuits. Table 1 below lists the time scales of neuronal activity and its significance.

TABLE 1

| Period | | Frequency Band | | Biological Significance |
|---|---|---|---|---|
| 100 s~100+ min | | <0.01 | Hz | Longer-term plasticity |
| | | | | mechanisms |
| 100 ms~100 s | | 0.01~10 | Hz | Local shorter-term |
| | | | | plasticity |
| 250~1000 | ms | 1~4 | Hz | Delta |
| 125~250 | ms | 4~8 | Hz | Theta |
| 80~125 | ms | 8~12.5 | Hz | Alpha |
| 40~80 | ms | 12.5-25 | Hz | Beta |
| 12.5~40 | ms | 25~80 | Hz | Low gamma |
| 5~12.5 | ms | 80~200 | Hz | High gamma |
| 0.1~5 | ms | 200~10000 | Hz | Membrane time constant, AP |
| | | | | (action potential) width, |
| | | | | max spike firing rates |

| | | |
|---|---|---|
| Beyond | Beyond | Likely smeared by membrane |
| | | time constants |

System 100 generates ultrasound waveforms whose timing parameters depend on the target brain regions. Similar to theta-burst TMS and other approaches, use of timing parameters consistent with a brain region's natural response can reduce the total treatment time compared to systems that rely on a more trial-and-error-based approach for the ultrasound waveform parameters, similar to pushing a swing at the right time, in phase and at the natural oscillation frequency. Alternatively, use of an unnatural timing (phase or frequency) at a brain region or structure can be used to disrupt or reduce ongoing activity levels, much like pushing a swing out of phase or at a frequency very removed from the natural oscillation frequency. System 100 generates frequencies and phases of the ultrasound waves aligned with the neuronal activity of the target region or regions, which can give a more effective energy coupling, i.e., transduction of ultrasound energy into neural activity. To maximize neuronal activity, the ultrasound waves generated by System 100 coincide with peaks of neuronal activity. Alternatively, they can coincide just before or after those peaks of neuronal activity or neuronal excitability to extend their activity duration. To inhibit neuronal activity, waveforms that are contrary to neuronal oscillations are generated by System 100. Such waveforms could be out of phase, constant, or at frequencies at which the target neuronal structures would typically not fire. The frequency bands in Table 1 are not demarked and could have overlaps. Using real-time feedback (for example, from EEG in PM 140, US imaging, evoked potentials, or other physiological measurements), System 100 can shift the frequency and phase of the waveforms generated to be faster or slower. For the waveforms that coincide within the frequency bands, System 100 can generate waveforms that are phase-locked to subject 105's oscillations of the target brain regions.

System 100 can generate layered or combined waveforms using amplitude modulation or various methods of gating or triggering of higher frequency bands by lower frequency bands. The combined waveforms use frequencies (or phases) from two or more frequency bands listed in Table 1 or from other feedback (e.g., EEG). For example, the waveform consists of a low tonic firing and another burst firing at a different frequency. The burst firing coincides with neuronal oscillation at a specific phase. System 100 can generate combined waveforms using gating, triggering, or other combining techniques. System 100 can adjust the duty cycle and phase of the waveforms generated.

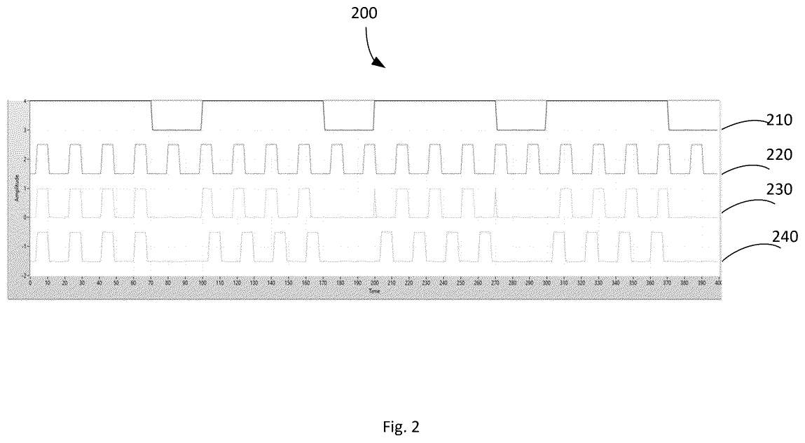

FIG. 2 shows example waveforms generated by System 100. Waveform 210 shows an example of a slow oscillation waveform with a period of 100 ms and a duty cycle of 70% (on time of 70 ms). Waveform 220 shows a fast oscillation with a period of 19 ms and a duty cycle of 31% (on time of 6 ms) with an offset of 4 ms (from waveform 210). Waveform 230 shows a gating waveform example, where waveform 230 results from the gating (logical “AND” operation) of waveform 210 and waveform 220. Waveform 230 has the same phase as the waveform 220. Waveform 240 is a triggering waveform example and results when waveform 210 triggers waveform 220. Waveform 240 starts when Waveform 210 turns on but with an offset of 4 ms. The second and third bursts of Waveform 230 and Waveform 240 highlight the differences between the gating and triggering waveforms.

### Criticality of US Waveforms & Field Shape

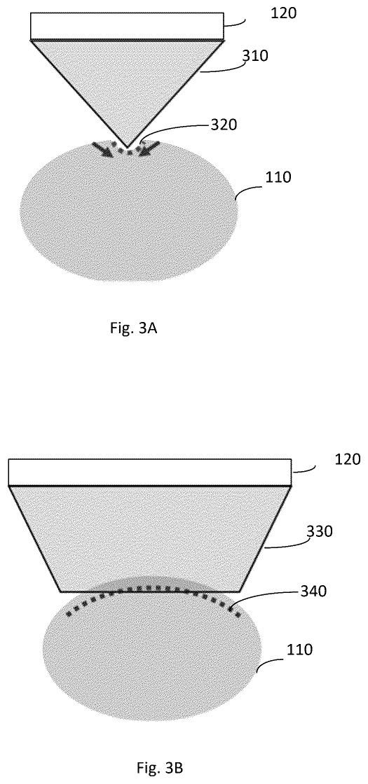

The use of a weakly focused waveform as shown in FIG. 3B will lead to compression of the tissue. However, such changes are not optimal for enacting neural activity at safe or reasonable pressures. In contrast, optimally configured ultrasound waveforms and fields without high peak intensities and pressures can effectively stretch neurons, thus opening ion channels or collapsing neurotransmitter vesicles into the presynaptic terminal's membrane and leading to neural activity or communication changes. FIGS. 3A and 3B show the effects of US waveforms with a low and high F-number, respectively. The focusing strength of an ultrasound beam can be expressed as an F-number or NA (numerical aperture). The following equation relates NA and F-number.

$$N A = ( \frac{1}{2 F} )$$

FIG. 3A shows an example of a low F-number field (high NA or tight focus). Transducer 120 in System 100 generates an ultrasound field 310, stretching the shallow cortex of the subject's head 110, resulting in strain 320. In FIG. 3B, an example of a high F-number field, transducer 120, generates an ultrasound field of 330. The result is shown as displacement 340. The power is the same in the examples; however, the lower F-number example in FIG. 3A creates more strain as the displacement of tissue is more spatially confined relative to the surrounding tissue. High F-number fields are ineffective in enacting changes in neuronal activity. Fields generated using a high F-number push down and move a broad region downward or compress it. In the shallow cortex especially (close to the ultrasound transducer), there are few mechanisms to enact neuron stretching. While local mechanisms relying just on pressure or intensity (local membrane cavitation, jet streaming, etc.) have been suggested as mechanisms for ultrasonic neuromodulation, negative results in large animals or in cases where standing waves or other confounds are properly minimized suggest that these mechanisms are not predominant underlying mechanisms for strong effects such as evoked motor responses. As TMS has shown us, even reliable and strong momentary stimulation does not easily lead to long lasting therapeutic effects, highlighting the criticality of being able to obtain strong, predictable, and reliable ultrasonic neuromodulation.

### Optimizing Pressure Spatial Derivative or Gradient

The waveform's intensity (peak pressure) is typically increased to improve modulation strength or reliability. This has limited efficacy. In addition, regulatory or safety constraints limit the maximum negative pressure excursion and maximum power deposition into tissue. Spatial pressure derivatives or other “gradients” can be high or low even for a given pressure and field shape. Increasing the center acoustic frequency of the stimulus increases the gradient in the axial direction, but the attenuation in the skull limits how high the center frequency can be set. Acoustic fields with higher spatial pressure derivatives give more efficacious neuromodulation. Acoustic fields without large spatial derivatives of pressure lack effective mechanisms for neuromodulation. Positive clinical results may stem from accidental misdirection of the focus, where the target structure experiences a “shoulder” of the beam with a significant pressure gradient because the focus is slightly offset. Or, if the target structure is larger than the beam focus, some parts of the structure will inevitably experience a spatial derivative of pressure since the “shoulders” of the beam will be within the structure. A higher pressure gradient translates to better stretching of brain regions and, hence, better neuromodulation. To achieve neuromodulation, a waveform, apodize function, aperture, or aperture combination that produces a higher value of a pressure spatial derivative is a better one. In an embodiment, for a given treatment volume (or target area), System 100 generates waveforms with optimized (maximizing) pressure gradient (M1, M2, or M3). This can be done by maximizing the ratio of the spatial derivative of the pressure to a given maximum pressure. In an embodiment, optimizing the apodize function (the variation in drive voltage amplitude supplied by Output Stage 174 to each transducer element) optimizes the pressure gradient. System 100 can also maximize the ratio using two or more apertures generating different frequencies with intersecting beams or ultrasound fields. The following equations show examples of System 100 optimizing the pressure gradient by maximizing one of the following ratios, M1, M2 or M3 of the pressure derivative to the maximum power.

$$M 3 = [ {( \frac{\partial P}{\partial x} )}^{2} + {( \frac{\partial P}{\partial y} )}^{2} + ( 1 / c ) {( \frac{\partial P}{\partial t} )}^{2} ] / {P}_{max}^{2}$$

$$M 2 = [ {( \frac{\partial P}{\partial x} )}^{2} + {( \frac{\partial P}{\partial y} )}^{2} ] / {P}_{max}^{2}$$

$$M 1 = [ {( \frac{\partial P}{\partial x} )}^{2} ] / {P}_{max}^{2}$$

Pₘₐₓ is the maximum pressure, and x, y, and t represent the three space coordinates where “x” is the lateral or medial direction, “y” is the anterior or posterior direction, and “t” is proportional to the superior or inferior direction. In ultrasound, the depth (superior or inferior) coordinate z is proportional to time via z=ct, where c is the sound speed in tissue. Therefore, the ∂t derivative can replace Oz. System 100 can optimize pressure gradient metric M3 in all three directions (x, y, z), metric M2 in two directions (x, y), or metric M1 one direction (x). This allows System 100 to appropriately control the direction in which the neurons, axons, or tracts move.

These metrics are normalized to the maximum pressure Pₘₐₓ in order to make the metrics independent of the absolute pressure applied. For neuromodulation purposes, fields and waveforms that maximize these metrics are preferred so that he lowest possible pressure can be selected for treatment.

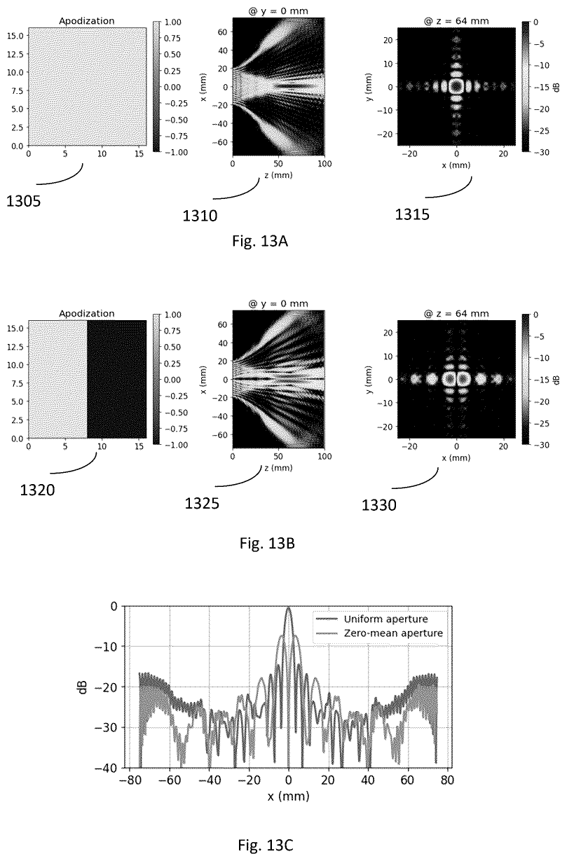

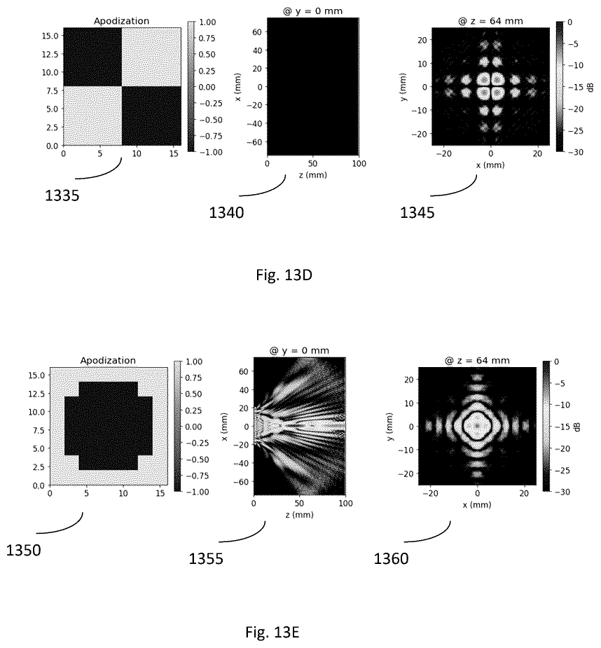

FIG. 13A-E shows example simulation results using apodization to increase gradient metrics. In the simulations the quantity P²ₘₐₓ used in the definition of metrics M1, M2 and M3 is held constant. All these simulations are done at the same frequency of 0.75 MHz, which means that the value of (∂P/∂t)² in the formula for the metric M3 is the same in each simulation. These simulations only show improvements in the spatial metrics M1 and M2 when the apodization function is altered.

FIG. 13A (prior art) shows a simulation of a 16×16 element ultrasound transducer. In this simulation, the apodization function 1305 is flat, meaning that all elements of the transducer are driven with the same amplitude. The plot 1310 shows the simulation field in depth (z) and laterally (x) with the y coordinate set to zero. A broadside focus can be seen as well as grating lobes around 45 degrees. Plot 1315 shows x and y directions at a fixed depth. This example is a common way of operating an ultrasound system, where there is no attempt to maximize the pressure gradient. This produces neuromodulation as a side effect and is inefficient in its usage of ultrasonic power.

FIG. 13B shows an apodize function which improves the field gradient in the lateral or x direction. Plot 1330 shows a rapid pressure change at and near x=0 for y coordinates close to y=0. The apodize function shown in 1320 produces a better M1 metric than the function shown in FIG. 13A.

FIG. 13C shows a graph comparing pressure magnitudes for the apodize functions used in FIGS. 13A and 13B. The graph shows the pressure plotted as a function of x with y=0. The spatial derivative dP/dx for FIG. 13B's apodize choice can be seen to be larger than that produced by FIG. 13A in the region around x=0.

FIG. 13D shows a simulation where the field gradient improves in both the x and y directions using apodize function shown in 1135. Plot 1345 shows spatial derivative is increased in x and y directions. The apodize function shown in plot 1335 improves M2. A consequence is that very little sound is produced in the y=0 plane shown in plot 1340.

FIG. 13E shows another example of an apodize function 1350 which improves metric M2.

### Anatomically Driven TUS Treatment Fields.

Brain structures are often connected to other regions via axons and other structures. In addition, the extracellular matrix inherently holds together brain cells and structures locally. When these other regions are not being displaced by ultrasound, we can consider them “anchored” due to their surrounding structures. These structures may be more distal structures connected to the target region via axons. They may be the target brain region while the surrounding non-target region can be considered anchored. They may also be the periphery of the target brain region if the ultrasound field is highly focused and only applying significant displacement to a portion of the target region. Analyzing these brain regions or structures, it is evident that moving the brain regions toward a connected, anchored brain region creates some slack (and not strain), leading to less efficacious neural modulation. Anatomically driven TUS treatment fields consider this anchoring of brain structures relative to their surrounding regions or other brain regions. This means a correctly directed field produces a radiation force that creates efficacious strain within the cell bodies or processes such as axons and dendrites, thus leading to more efficacious neural modulation. The following sections provide examples of System 100 generating treatment fields driven by the target volume's anatomy.

### Stretching Along Axon or WM Tract

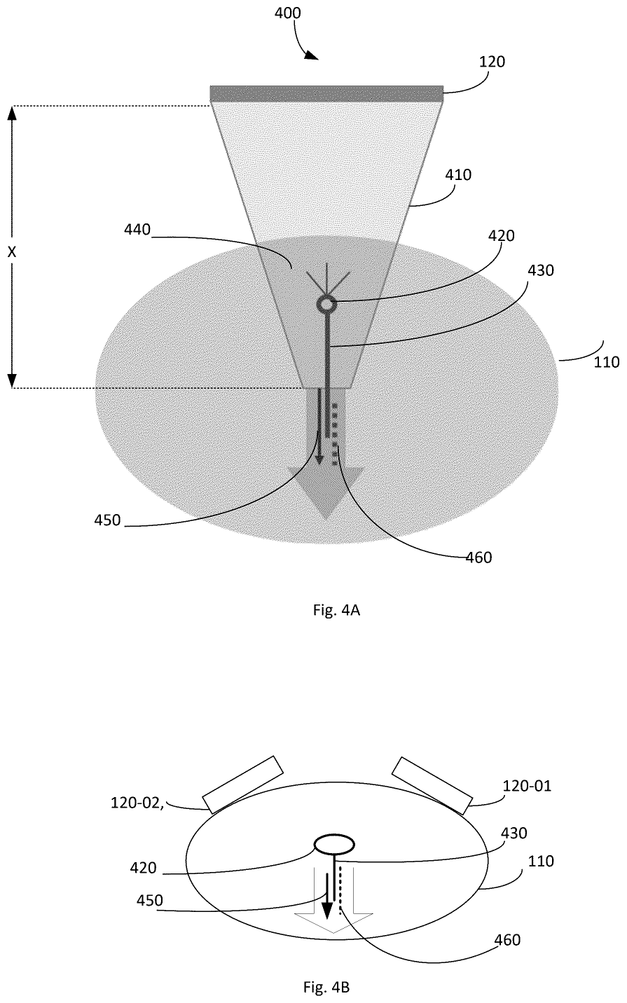

There is quite a bit of variability in brain regions, but some regions have ordered (or nearly ordered) layers of neurons, axonal tracts, etc. There may even be differences in a single brain region, with the output neurons being ordered while local interneurons may not be. In an embodiment, System 100 aims to maximally stretch an axon or white matter tract by aligning the ultrasound propagation along the orientation of the neural target (axon or WM). If the axial depth of focus is too long and the aim is too shallow, it will simply push the entire structure along (the whole neuron, brain region, and white matter, etc.). Thus, System 100 keeps the axial depth of focus to fall within the axon/WM or maximize ARF (acoustic radiation force) at some point beyond the cell body, within the axon or at the WM tracts to stretch the neuron. This approach relies on highly ordered brain regions or at least a population of neurons with this ordering and directionality. FIG. 4A shows an example of System 100 creating stretching along an axon or WM tract. The example shows neuron 420 with axon 430 and dendrites 440 (or dendritic tree). Aperture 120 of System 100 generates an ultrasound field 410, where the axial depth (shown as X) of focus falls beyond neuron 420 and within axon 430. This creates an ARF (depicted by the wider arrow) that maximally falls beyond the cell body and within axon 430. The resultant longitudinal strain 450 stretches the structure, which is shown as 460. To maximize the ARF at a depth beyond neuron 420 (or the group of cell bodies) relative to the displacement of the shallower portions (e.g. cell bodies), System 100 uses a low F-number. This example shows a single neuron and axon, and this can be extended for white matter tracts, which are aligned axon bundles. Skull reflections limit how low the F-number can be reduced. To overcome this, using two or more apertures 120 is advantageous, as shown in FIG. 4B. Apertures 120-01 and 120-02 are placed at different locations on the subject's head 110. Like the example in FIG. 4A, the two apertures (more than two can be used) move the structures (460) differentially relative to more “anchored” portions, thus creating strain (450). The relative angle between the transducers 120-01 and 120-02 can be adjusted per an embodiment of the system.

### Stretching Orthogonal to Dendritic Tree

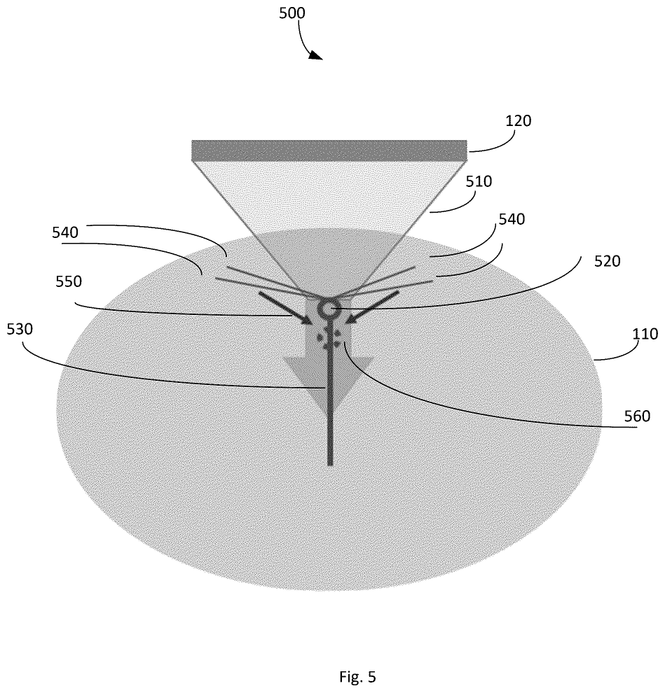

If dendrites are anchored well (for example, they spread a large distance laterally within the brain region in a relatively ordered manner), the cell body can be pushed down to stretch the neuron. If the dendrites branch out widely in a brain region, they are like a net that anchors them. If they are a dendritic tree that spans multiple millimeters, it would be possible to have a narrower beam (say, 1 mm diameter) to push a column of cell bodies downward, stretching them relative to the dendrites. FIG. 5 shows an example of System 100 displacing a cell body attached to a well-anchored dendritic tree, leading to stretching of the structures. Neuron 520 with axon 530 is attached to a well-anchored dendritic tree 540. Aperture 120 of System 100 generates an ultrasound field 510 (or beam), where the axial depth of focus falls on neuron 520. This creates an ARF (depicted by the wider arrow). The resultant strain 550 stretches the structure, which is shown as 560. In this case again, higher NA is useful. A low NA (“flat wavefront”) will simply push down on the dendrites and cell bodies together, much like in the displacement case of FIG. 3B, as opposed to significant local deformation.

### Stretching Orthogonal to Axon or WM Tract

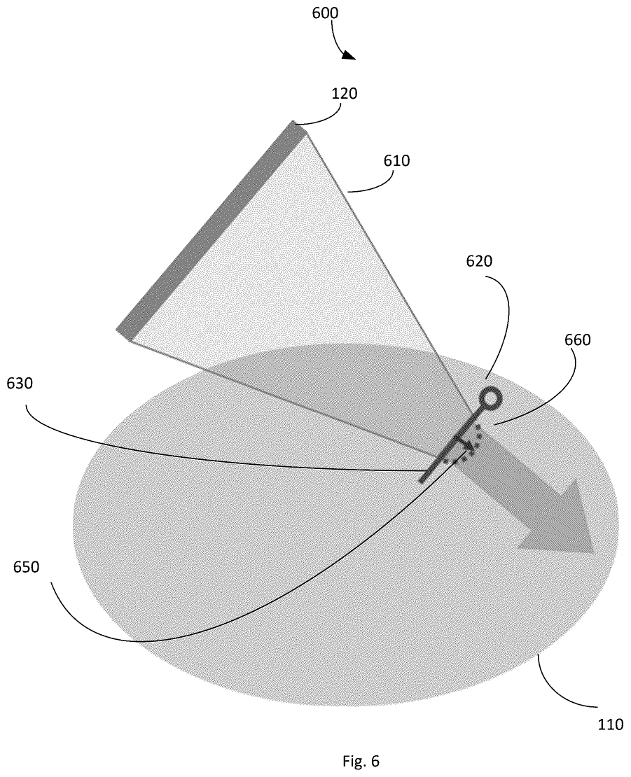

Similar to the longitudinal stretching shown in FIG. 4A, System 100 can generate fields that are orthogonal to axons and white matter tracts. This produces an effect similar to plucking a guitar string. FIG. 6 shows an example of System 100 generating a field that generates orthogonal stretching of an axon. Axon 630 is attached to Neuron 620. Aperture 120 of System 100 generates an ultrasound field 610 (or beam), where the field is orthogonal to axon 620. This creates an ARF (depicted by the wider arrow). The resultant strain 650 stretches the structure, which is shown as 660.

### Antiparallel, Spatially Limited Standing Waves

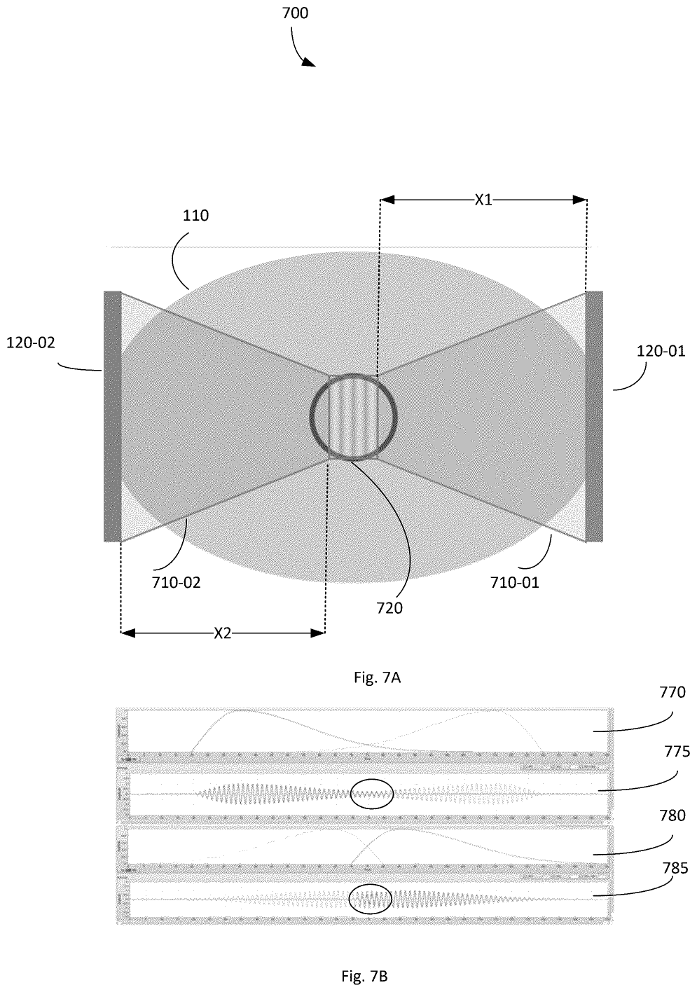

Standing waves can create spatially extended fields, especially with relatively long focal zones that span multiple wavelengths. System 100 leverages the natural axial focal depth of US fields by aiming a bit short or long so that the overlapping regions where standing waves form are limited in space. Angling of the wavefronts can limit the spatial extent. FIG. 7A shows an example of System 100 using antiparallel, spatially limited standing waves for modulation. The target region of the subject's head 110 is shown as circle 720. In this example, two transducers, 120-01 and 120-02, are placed opposite each other, with their fields shown as 710-01 and 710-02, respectively. The ultrasound fields' focal depths (marked as X1 and X2) are aimed short. The oppositely placed transducers 120 create standing waves shown as banded lines within target 720. The short focal depth of the transducers limits the spatial extent of standing waves.

FIG. 7B shows simulation graphs of antiparallel spatially limited standing waves. Graph 770 shows the pressure envelopes produced by the transducers 120-01 and 120-02. Graph 775 shows a snapshot in time of the two pressure waveforms. The horizontal axis is time, proportional to the distance to the focal point from the transducer. The y-axis plots the amplitude. The standing waves (marked by the circle on Graph 775) are limited to the extent of the pressure envelope overlap, as shown in Graph 770. Graph 780 shows the envelope of the pressure fields with the transducer foci at different positions from Graph 770. The resulting interference pattern causing standing waves is shown in Graph 785.

Standing waves can also be created by reflections. For example, for the shallow cortex relative to the skull/brain but distal from the transducer, reflections from the skull can create standing waves. However, simply having a low NA field that reflects from a skull “head-on” is not ideal. Again, such methods can lead to rather large areas (spanning multiple wavelengths) of neuromodulation. Choosing the correct angle of the wavefront (so that the incident angle on the skull leads to a relatively small region of standing waves near the skull), NA (large NA for shorter focal zones, leading to shorter regions of standing waves), and other parameters creates standing waves with limited spatial extent.

Thus, if standing waves are to be used as a mechanism for ultrasonic neuromodulation, methods to reduce their spatial extent are preferred to reduce off-target modulation or other unintended effects.

### Antiparallel Stretching

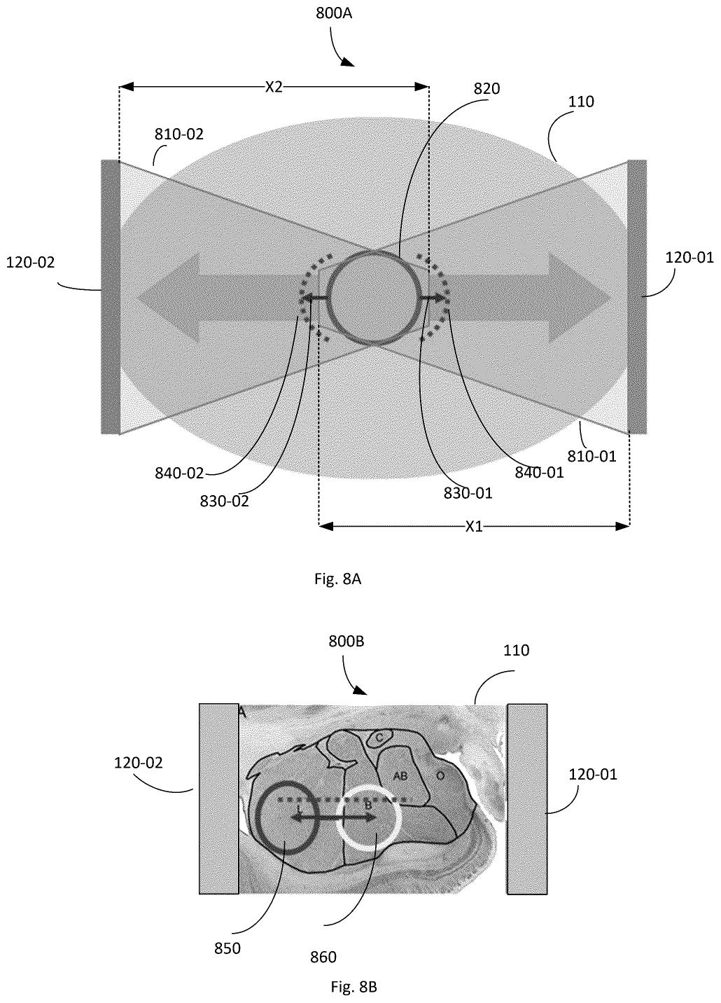

In contrast to using standing waves, in an embodiment, System 100 uses two (or more) transducers focused beyond each other, imparting pushing forces on the other side, leading to stretching. Slight angling of these two wavefronts or using distinct frequencies can reduce or eliminate standing waves while imparting effective stretching of the brain region. This mechanism and approach do not require frequency sweeping or other methods to alter or reduce standing waves. FIG. 8A shows an example of System 100 generating antiparallel single region stretching. The target region of the subject's head 110 is shown as circle 820. In this example, two transducers, 120-01 and 120-02, are placed opposite each other, with their fields shown as 810-01 and 810-02, respectively. The ultrasound fields' focal depths (marked as X1 and X2) are aimed beyond the target 820. The oppositely placed transducers 120 create strains 840-01 and 840-02, resulting in stretching of the target 820 (shown as 840-01 and 840-02). Having an angle between the transducer 120-01 and 120-02 (not directly opposite to each other) or operating them at different frequencies ensures that there are no standing waves.

Similarly, System 100 can be used for antiparallel stretching to separate two brain regions or the connections between two brain regions. In FIG. 8B, brain regions 850 and 860 are being stretched apart using transducers 120-01 and 120-01. This stretching can be useful for regions connected more locally than those that go through WM tracts.

Stretch and Strain within a Single Brain Region

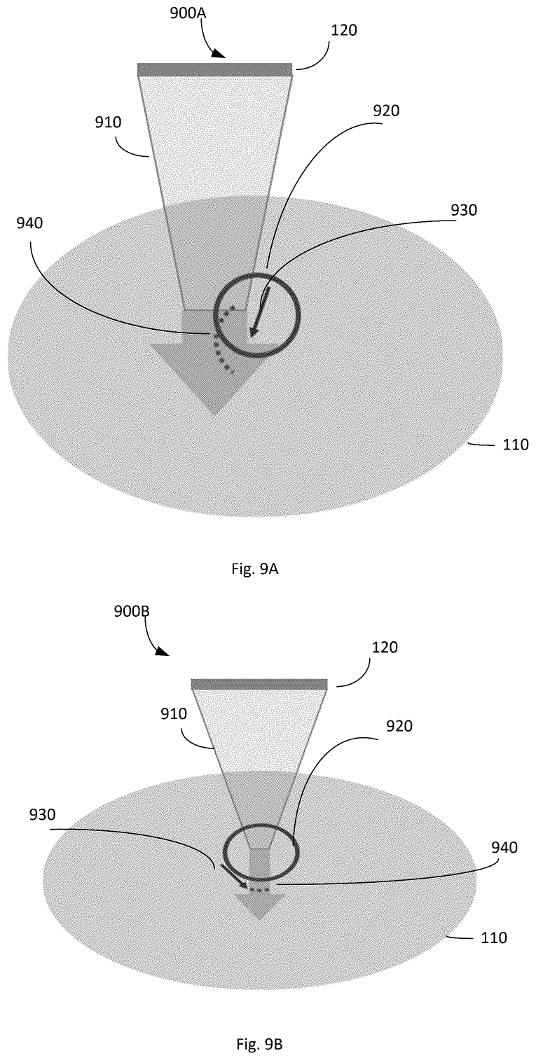

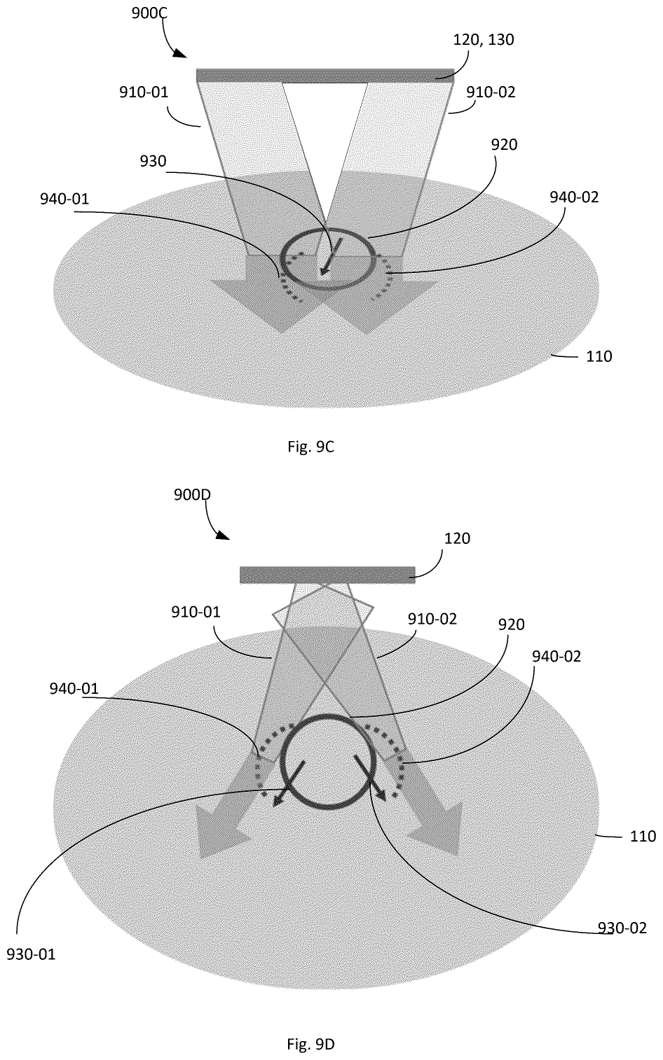

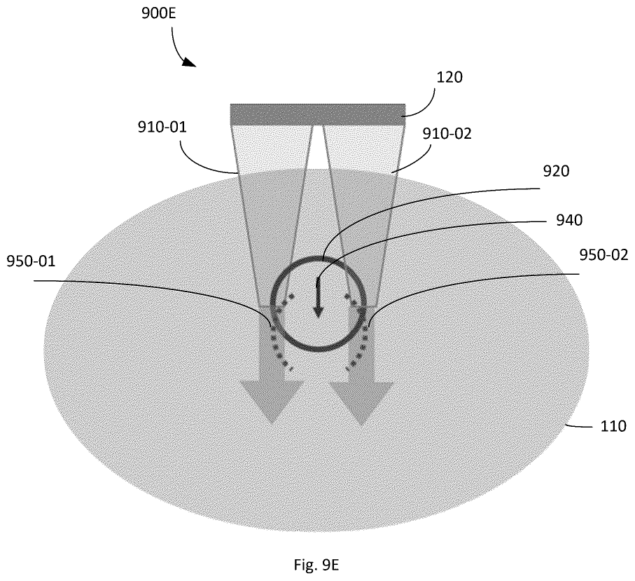

In an embodiment, System 100, with one or more apertures acting on the edges of a single brain region, can stretch that region. System 100 can direct ultrasound wavefronts on one or more sides of the target alternately or simultaneously. The angle at which the wavefront approaches the target can be modified. FIG. 9A shows an example of System 100 creating stretching and strain within a single brain region. Transducer 120 within System 100 creates a narrow wavefront 910 to one side of the target brain region 920 to create shearing. The resultant shearing is shown as 940. Similarly, in FIG. 9B, System 100 pushes down on the middle of a narrow target 920 with a narrow beam 910 with transducer 120. In FIG. 9C, two apertures (from one or more transducers) aim at the two sides of the target 920 alternately, causing the stretching to cycle between the left (940-01) and right sides (940-02). In FIG. 9D, simultaneous or alternate stretching of sides of the brain region 920 can be performed by steering the wavefront. The alternate steering is shown as 910-01 (going toward the left of the target 920) and 910-02 (towards the right). A single aperture 120 is shown in this example. In FIG. 9E, simultaneous stretching of the edges in a ‘non-glancing’ angle approach is performed.

Maximizing Stretching of Processes from and to Deeper Regions

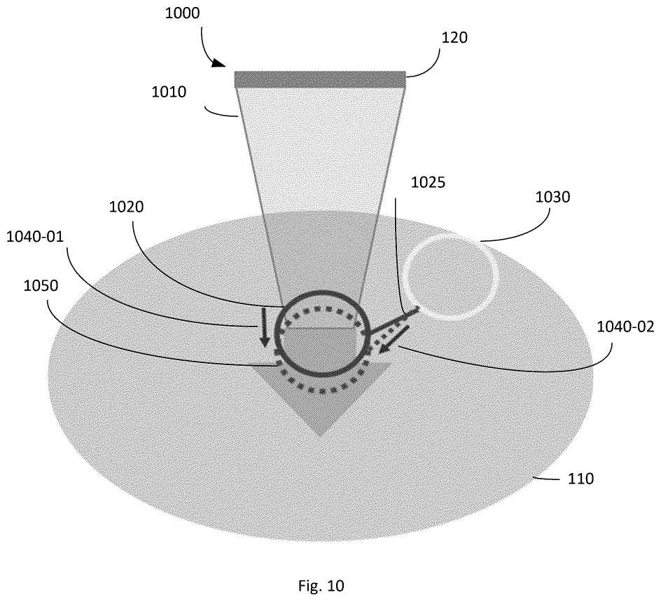

In an embodiment, System 100 targets deeper brain regions with axons or WM tracts from “above” (relative to the direction of ultrasound propagation) to stretch to modulate the brain region inputs or outputs. The activation of outgoing axons or tracts can lead to backpropagated action potentials that activate neurons in the target brain region. FIG. 10 shows an example of System 100 targeting tracts from or to deeper brain regions. Deep target region 1020 is connected to a shallower region 1030 via tract 1025. Transducer 120 generates an ultrasound field or beam 1010 focused directly on target 1020. This creates a force 1040-01 to push the target towards 1050. The moving of target 1020 towards 1050 causes the tract 1025 connecting to region 1030 to stretch and thus activate neurons in the brain regions.

### Collinear Targeting of Two Regions

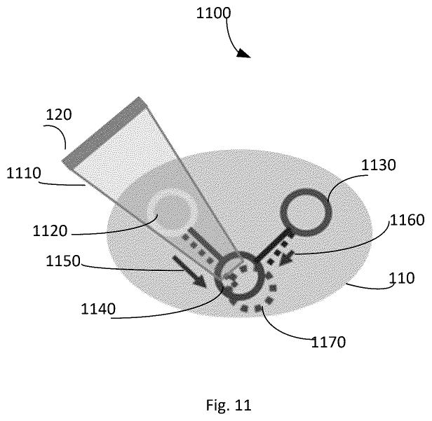

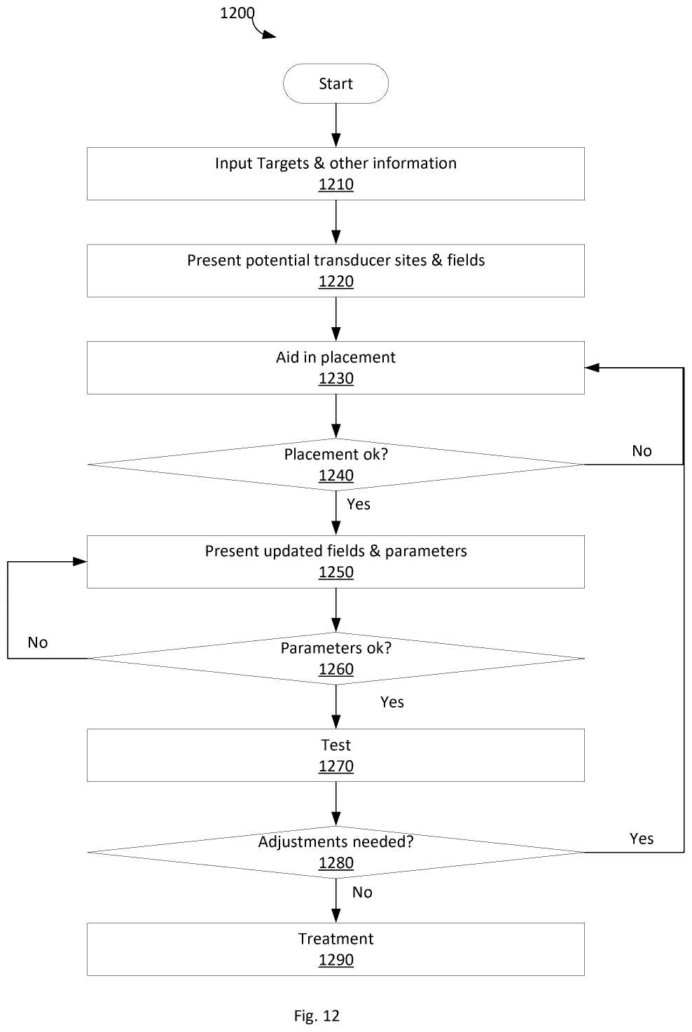

The preceding targeting approach modulates the target brain region relatively non-specifically with regard to connected regions. System 100 can target regions that are connected to multiple regions with higher selectivity. Collinear targeting allows System 100 to modulate specific connections. FIG. 11 shows an example of System 100 collinear targeting of two regions. Brain region 1140 is connected to regions 1120 and 1130. The goal of the System is to modulate the tracts between regions 1120 and 1140 and not (or minimize) the modulation of the tracts between regions 1140 and 1130. Transducer or aperture 120 generates a collinear field 1110, where the direction of the force (1140) generated moves region 1140 towards 1170 to modulate the tracts between 1120 and 1140. Due to the collinear targeting of regions 1120 and 1140, there is minimal or no movement of the tract between 1140 and 1130. Note if a transducer 120 is positioned above region 1140, similar to FIG. 10, this would simply push down region 1140 and create stretching of both the tracts or connections.

Method for Ultrasound Parameters based on Target anatomy

FIG. 12 shows an example of method 1200 using System 100 for transcranial stimulation. Method 1200 provides suggested transducer placement locations and ultrasound parameters based on a clinician's input of the target stimulation regions. It aids the clinician to visualize the ultrasound fields and the resultant stretching. The clinician can then quickly modify the ultrasound parameters and visualize the changes. Method 1200 starts in Operation 1210, where the clinician or physician inputs the target brain region. Clinician 190 enters the information using UI 180. Clinician 190 may enter other pertinent information, such as the unique physical characteristics of patient 105, locations on patient 105's head to avoid, preferred probe 120 locations, other ultrasound parameters, etc. Other information, like the patient's MRI data, EEG or other physiological measurement data, patient head measurements, or results from previous neuromodulation sessions, etc., may be provided to System 100.

In Operation 1220, Method 1200 computes various stimulation parameters. This includes the number of transducers 120 for stimulation, imaging, or stimulation and imaging, placement locations for the transducers 120, etc. System 100 in this Operation also displays the ultrasound fields to Clinician 190 and presents updated target information. For example, System 100 may suggest focusing on the lateral sides of the target region and thus having two target locations for the ultrasound fields. When System 100 is used for two connected brain regions, especially if the distance between the regions is large, the area of stretching could be in several areas between the two regions. In this scenario, Method 1200 suggests parameters to minimize off-target effects. In general, Method 1200 optimizes the parameters to minimize off-target effects. Method 1200 provides suggested timing parameters based on the time scale of the neuronal activity of the target brain region. Method 1200 optimizes the pressure gradient to maximize the stretching. Method 1200 could optimize the pressure gradient in one, two, or three directions. The directions correspond to the three axes: x, y, and z.

In Operation 1230, Method 1200 aids clinician 190 in placing transducers 120 on the patient's head 110. Method 1200 uses neuronavigational equipment and provides aids (such as a display on UI 180) to guide clinician 190 in placing the transducers 120. Method 1200 may also utilize physiological measurements 140 (such as EEG) and imaging data from one or more transducers 120. Clinician 190 places transducers 120 on the patient's head 110.

In Operation 1240, the placements of transducers 120 are evaluated. If transducer placements are not optimal, Method 1200 continues aiding Clinician 190 in Operation 1230. Otherwise, Method 1200 presents updated fields and ultrasound parameters for Clinician 190 to evaluate in Operation 1250.

Throughout the operations, in particular Operations 1220, 1230, and 1240, ultrasound field shape simulation and aberration correction algorithms may be used. The algorithms account in particular for field distortions such as lateral displacements; altering of the locations of maximal pressure, pressure gradients, or other parameters of the field; and defocusing or other smearing of the field affecting the efficacious parameters that happen due to the presence of the skull. In 1220, they may be used to suggest initial optimal placements. In 1230 and 1240, they may use real time location of the transducer as well as ultrasonic imaging and measurements to further refine the aberration corrections.

In Operation 1260, Clinician 190 evaluates the ultrasound parameters and makes necessary adjustments. Method 1200 reverts to Operation 1250. If parameters and fields are okay, Method 1200 proceeds to operation 1270.

In Operation 1270, System 100 uses test pulses and waveforms to evaluate the placement, fields, and other ultrasound parameters. This Operation allows for any corrections, for example, caused by skull aberrations, reflections, or any required changes to foci, etc. Several strategies are employed for this Operation. For example, a first transducer emits test waveforms, and the results are evaluated by a second (or more than one) transducer. In a different example, the receive elements of the first transducer are used for imaging and evaluating the results. Physiological measurements 140 using EEG or EMG are used in a different example. Evoked potentials are used in a different example.

In Operation 1280, results from Operation 1270 are evaluated. Clinician 190, using the aid of System 100, determines whether changes or adjustments to the placements of transducers 120 or other ultrasound parameters are required. For example, based on the timing of neuronal activity of the target region (measured using EEG 140), the frequency and phase of the ultrasound waveforms need modification. System 100 may phase lock to the signal detected by EEG 140.

Once adjustments are complete, Method 1200 proceeds to Operation 1290, where Subject 105 is treated or stimulated.

FIG. 12 illustrates Method 1200 as a flow chart, with each Operation shown to be distinct. Multiple Operations can be combined into a single Operation; for example, successive Operations may be combined into a Single Operation.
1. In some embodiments, a system comprises at least one transducer, a physiological measurement system, and at least one processor executing an application, wherein the application causes the at least one processor to at least perform the steps of receiving a selection of at least one target anatomy, computing a plurality of ultrasound parameters for the at least one target anatomy based upon a selected treatment and the at least one target anatomy, generating a placement and angular pose of the at least one transducer, and causing the at least one transducer to apply stimulation to the at least one target anatomy.
2. The system of clause 1, wherein generating the placement and angular pose of the at least one transducer is based on real-time feedback from the physiological measurement system, neuronavigational data from a neuronavigation subsystem, or transducer imaging data from the at least one transducer.
3. The system of clauses 1 or 2, wherein causing the at least one transducer to apply stimulation further comprises generating an ultrasound waveform phase or frequency locked to waveforms detected by the physiological measurement system.
4. The system of any of clauses 1-3, wherein the application refines the ultrasound parameters based on real-time data from the physiological measurement system, evoked potentials, or transducer imaging data from the at least one transducer.
5. The system of any of clauses 1-4, wherein the application refines the ultrasound parameters and timing to correct skull aberrations.
6. The system of any of clauses 1-5, wherein causing the at least one transducer to apply stimulation using the at least one transducer comprises causing the at least one transducer to generate an ultrasound radiation force that is directed along the axonal or white matter tracts and is maximized beyond the average distance of the cell bodies from the transducers to stretch axons or white matter tracts.
7. The system of any of clauses 1-6, wherein causing the at least one transducer to apply stimulation comprises causing the at least one transducer to generate an ultrasound radiation force that is directed to stretch a cell body attached to an anchored dendritic tree.
8. The system of any of clauses 1-7, wherein causing the at least one transducer to apply stimulation comprises causing the at least one transducer to generate an ultrasound radiation force that is directed orthogonal to axons and white matter tracts.
9. The system of any of clauses 1-8, wherein the at least one transducer comprises two or more transducers placed opposite to each other and the application causes two or more transducers to generate spatially limited standing waves by generating focal depths short of the at least one target anatomy.
10. The system of any of clauses 1-9, wherein the two or more transducers use anti-parallel stretching of target anatomy by generating ultrasound radiation force fields aimed beyond the at least one target anatomy.
11. The system of any of clauses 1-10, wherein the application causes the at least one transducer to generate ultrasound radiation forces on one or more sides of the at least one target anatomy alternately or simultaneously.
12. The system of any of clauses 1-11, wherein the application causes the at least one transducer to generate an ultrasound radiation force that is directed to stretch axons or white matter tracts to or from deeper brain regions.
13. The system of any of clauses 1-12, wherein the application causes the at least one transducer to generate a collinear ultrasound radiation force to target a multiple connected brain region to stretch in a specific region of the at least one target anatomy.
14. In some embodiments, a method comprises receiving a selection of at least one target anatomy, computing a plurality of ultrasound parameters for the at least one target anatomy based upon a selected treatment and the at least one target anatomy, generating a placement and angular pose of at least one transducer, aiding in the placement of the at least one transducer, identifying an adjustment to the placement of the at least one transducer, and causing the at least one transducer to apply stimulation to the at least one target anatomy.
15. The method of clause 14, wherein aiding the placement further comprises causing the at least one transducer to emit at least one test pulse or at least one waveform, wherein the adjustment is determined based upon a response to the at least one test pulse or at least one waveform.
16. The method of clauses 14 or 15, wherein identifying an adjustment to the placement of the at least one transducer is based on real-time feedback from a physiological measurement system, neuronavigational data from a neuronavigation subsystem, or transducer imaging data from the at least one transducer.
17. The method of any of clauses 14-16, wherein causing the at least one transducer to apply stimulation further comprises generating an ultrasound waveform phase or frequency locked to waveforms detected by a physiological measurement system.
18. The method of any of clauses 14-17, wherein the method a refines the ultrasound parameters of the at least one transducer based upon real-time data from a physiological measurement system, evoked potentials, or transducer imaging data from the at least one transducer.
19. The method of any of clauses 14-18, wherein the method adjusts ultrasound parameters and timing of the at least one transducer to correct skull aberrations.
20. The method of any of clauses 14-19, wherein causing the at least one transducer to apply stimulation using the at least one transducer comprises causing the at least one transducer to generate an ultrasound radiation force that is directed along the axonal or white matter tracts and is maximized beyond the average distance of the cell bodies from the transducers to stretch axons or white matter tracts.
21. The method of any of clauses 14-20, wherein causing the at least one transducer to apply stimulation comprises causing the at least one transducer to generate an ultrasound radiation force that is directed to stretch a cell body attached to an anchored dendritic tree.
22. The method of any of clauses 14-21, wherein causing the at least one transducer to apply stimulation comprises causing the at least one transducer to generate an ultrasound radiation force that is directed orthogonal to axons and white matter tracts.
23. The method of any of clauses 14-22, wherein the at least one transducer comprises two or more transducers placed opposite to each other and the method further comprises causing two or more transducers to generate spatially limited standing waves by generating focal depths short of the at least one target anatomy.
24. The method of any of clauses 14-23, wherein the two or more transducers use anti-parallel stretching of target anatomy by generating ultrasound radiation force fields aimed beyond the at least one target anatomy.
25. The method of any of clauses 14-24, further comprising causing the at least one transducer to generate ultrasound radiation forces on one or more sides of the at least one target anatomy alternately or simultaneously.
26. The method of any of clauses 14-25, further comprising causing the at least one transducer to generate an ultrasound radiation force that is directed to stretch axons or white matter tracts to or from deeper brain regions.
27. The method of any of clauses 14-26, further comprising causing the at least one transducer to generate a collinear ultrasound radiation force to target a multiple connected brain region to stretch in a specific region of the at least one target anatomy.
28. In some embodiments, a system comprises at least one transducer, a physiological measurement system, and at least one processor executing an application, wherein the application causes the at least one processor to at least perform the steps of receiving a selection of at least one target anatomy, computing a plurality of ultrasound parameters for the at least one target anatomy based upon a selected treatment and the at least one target anatomy, wherein one of the ultrasound parameters comprises a pressure gradient optimized for a treatment volume associated with the at least one target anatomy, generating a placement of the at least one transducer, and causing the at least one transducer to apply stimulation to the at least one target anatomy.
29. The system of clause 28, wherein the pressure gradient is optimized by maximizing the ratio of a pressure space derivative to a maximum power of the stimulation.
30. The system of clauses 28 or 29, wherein the pressure space derivative is optimized in the lateral or medial, anterior or posterior, and superior or inferior directions.
31. The system of any of clauses 28-30, wherein the pressure space derivative is optimized in the lateral or medial and anterior or posterior directions.
32. The system of any of clauses 28-31, wherein the pressure space derivative is optimized in the lateral or medial direction.
33. The system of any of clauses 28-32, further comprising at least two apertures for generating tone bursts at different frequencies where fields of at least two apertures intersect to optimize the pressure gradient for a treatment volume associated with the at least one target anatomy.
34. The system of any of clauses 28-33, wherein the pressure gradient is optimized by maximizing the ratio of the pressure space derivative to the maximum power.
35. The system of any of clauses 28-34, wherein the pressure space derivative is optimized in the lateral or medial, anterior or posterior, and superior or inferior directions.
36. The system of any of clauses 28-35, wherein the pressure space derivative is optimized in the lateral, medial, anterior, or posterior directions.
37. The system of any of clauses 28-36, wherein the pressure space derivative is optimized in the lateral or medial direction.
38. In some embodiments, a system comprises at least one transducer, a physiological measurement system, and at least one processor executing an application, wherein the application causes the at least one processor to at least perform the steps of receiving a selection of at least one target anatomy, computing a plurality of ultrasound parameters for the at least one target anatomy based upon a selected treatment and the at least one target anatomy, wherein one of the ultrasound parameters comprises one or more biological factors determining a frequency or phase of a stimulation generated by the at least one transducer, generating a placement of the at least one transducer, and causing the at least one transducer to apply stimulation to the at least one target anatomy.
39. The system of clause 38, wherein the one or more biological factors comprise timing parameters of the brain region or regions being targeted or their connections.
40. The system of clauses 38 or 39, wherein the one or more biological factors comprise neural activity timing.
41. The system of any of clauses 38-40, wherein feedback from the physiological measurement system is used to modify the ultrasound waves generated.
42. The system of any of clauses 38-41, wherein the stimulation comprises ultrasound waves phase-locked to a subject's natural oscillations of the target brain region or regions.
43. The system of any of clauses 38-42, wherein the stimulation comprises ultrasound waves generated using amplitude modulation of two or more frequencies.
44. The system of any of clauses 38-43, wherein the stimulation comprises waveforms generated from the gating of two or more waveforms.
45. The system of any of clauses 38-44, wherein the stimulation comprises waveforms generated from the triggering of two or more waveforms.
46. The system of any of clauses 38-45, wherein the waveforms are phase offset from one or more frequencies.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, for example, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A system comprising:
at least one transducer;
a physiological measurement system; and
at least one processor executing an application, wherein the application causes the at least one processor to at least perform the steps of:
receiving a selection of at least one target anatomy;
computing a plurality of ultrasound parameters for the at least one target anatomy based upon a selected treatment and the at least one target anatomy;
generating a placement and angular pose of the at least one transducer; and
causing the at least one transducer to apply stimulation to the at least one target anatomy.

2. The system of claim 1, wherein generating the placement and angular pose of the at least one transducer is based on real-time feedback from the physiological measurement system, neuronavigational data from a neuronavigation subsystem, or transducer imaging data from the at least one transducer.

3. The system of claim 1, wherein causing the at least one transducer to apply stimulation further comprises generating an ultrasound waveform phase or frequency locked to waveforms detected by the physiological measurement system.

4. The system of claim 1, wherein the application refines the ultrasound parameters based on real-time data from the physiological measurement system, evoked potentials, or transducer imaging data from the at least one transducer.

5. The system of claim 1, wherein the application refines the ultrasound parameters and timing to correct skull aberrations.

6. The system of claim 1, wherein causing the at least one transducer to apply stimulation using the at least one transducer comprises causing the at least one transducer to generate an ultrasound radiation force that is directed along the axonal or white matter tracts and is maximized beyond the average distance of the cell bodies from the transducers to stretch axons or white matter tracts.

7. The system of claim 1, wherein causing the at least one transducer to apply stimulation comprises causing the at least one transducer to generate an ultrasound radiation force that is directed to stretch a cell body attached to an anchored dendritic tree.

8. The system of claim 1, wherein causing the at least one transducer to apply stimulation comprises causing the at least one transducer to generate an ultrasound radiation force that is directed orthogonal to axons and white matter tracts.

9. The system of claim 1, wherein the at least one transducer comprises two or more transducers placed opposite to each other and the application causes two or more transducers to generate spatially limited standing waves by generating focal depths short of the at least one target anatomy.

10. The system of claim 9, wherein the two or more transducers use anti-parallel stretching of target anatomy by generating ultrasound radiation force fields aimed beyond the at least one target anatomy.

11. The system of claim 1, wherein the application causes the at least one transducer to generate ultrasound radiation forces on one or more sides of the at least one target anatomy alternately or simultaneously.

12. The system of claim 1, wherein the application causes the at least one transducer to generate an ultrasound radiation force that is directed to stretch axons or white matter tracts to or from deeper brain regions.

13. The system of claim 1, wherein the application causes the at least one transducer to generate a collinear ultrasound radiation force to target a multiple connected brain region to stretch in a specific region of the at least one target anatomy.

14. A method comprising:
receiving a selection of at least one target anatomy;
computing a plurality of ultrasound parameters for the at least one target anatomy based upon a selected treatment and the at least one target anatomy;
generating a placement and angular pose of at least one transducer;
aiding in the placement of the at least one transducer;
identifying an adjustment to the placement of the at least one transducer; and
causing the at least one transducer to apply stimulation to the at least one target anatomy.

15. The method of claim 14, wherein aiding the placement further comprises causing the at least one transducer to emit at least one test pulse or at least one waveform, wherein the adjustment is determined based upon a response to the at least one test pulse or at least one waveform.

16. The method of claim 14, wherein identifying an adjustment to the placement of the at least one transducer is based on real-time feedback from a physiological measurement system, neuronavigational data from a neuronavigation subsystem, or transducer imaging data from the at least one transducer.

17. The method of claim 14, wherein causing the at least one transducer to apply stimulation further comprises generating an ultrasound waveform phase or frequency locked to waveforms detected by a physiological measurement system.

18. The method of claim 14, wherein the method a refines the ultrasound parameters of the at least one transducer based upon real-time data from a physiological measurement system, evoked potentials, or transducer imaging data from the at least one transducer.

19. The method of claim 14, wherein the method adjusts ultrasound parameters and timing of the at least one transducer to correct skull aberrations.

20. The method of claim 14, wherein causing the at least one transducer to apply stimulation using the at least one transducer comprises causing the at least one transducer to generate an ultrasound radiation force that is directed along the axonal or white matter tracts and is maximized beyond the average distance of the cell bodies from the transducers to stretch axons or white matter tracts.

21. The method of claim 14, wherein causing the at least one transducer to apply stimulation comprises causing the at least one transducer to generate an ultrasound radiation force that is directed to stretch a cell body attached to an anchored dendritic tree.

22. The method of claim 14, wherein causing the at least one transducer to apply stimulation comprises causing the at least one transducer to generate an ultrasound radiation force that is directed orthogonal to axons and white matter tracts.

23. The method of claim 14, wherein the at least one transducer comprises two or more transducers placed opposite to each other and the method further comprises causing two or more transducers to generate spatially limited standing waves by generating focal depths short of the at least one target anatomy.

24. The method of claim 23, wherein the two or more transducers use anti-parallel stretching of target anatomy by generating ultrasound radiation force fields aimed beyond the at least one target anatomy.

25. The method of claim 14, further comprising causing the at least one transducer to generate ultrasound radiation forces on one or more sides of the at least one target anatomy alternately or simultaneously.

26. The method of claim 14, further comprising causing the at least one transducer to generate an ultrasound radiation force that is directed to stretch axons or white matter tracts to or from deeper brain regions.

27. The method of claim 14, further comprising causing the at least one transducer to generate a collinear ultrasound radiation force to target a multiple connected brain region to stretch in a specific region of the at least one target anatomy.

28. A system comprising:
at least one transducer;
a physiological measurement system; and
at least one processor executing an application, wherein the application causes the at least one processor to at least perform the steps of:
receiving a selection of at least one target anatomy;
computing a plurality of ultrasound parameters for the at least one target anatomy based upon a selected treatment and the at least one target anatomy, wherein one of the ultrasound parameters comprises a pressure gradient optimized for a treatment volume associated with the at least one target anatomy;
generating a placement of the at least one transducer; and
causing the at least one transducer to apply stimulation to the at least one target anatomy.

29. The system of claim 28, wherein the pressure gradient is optimized by maximizing the ratio of a pressure space derivative to a maximum power of the stimulation.

30. The system of claim 29, wherein the pressure space derivative is optimized in the lateral or medial, anterior or posterior, and superior or inferior directions.

31. The system of claim 29, wherein the pressure space derivative is optimized in the lateral or medial and anterior or posterior directions.

32. The system of claim 29, wherein the pressure space derivative is optimized in the lateral or medial direction.

33. The system of claim 28, further comprising at least two apertures for generating tone bursts at different frequencies where fields of at least two apertures intersect to optimize the pressure gradient for a treatment volume associated with the at least one target anatomy.

34. The system of claim 33, wherein the pressure gradient is optimized by maximizing the ratio of the pressure space derivative to the maximum power.

35. The system of claim 33, wherein the pressure space derivative is optimized in the lateral or medial, anterior or posterior, and superior or inferior directions.

36. The system of claim 33, wherein the pressure space derivative is optimized in the lateral, medial, anterior, or posterior directions.

37. The system of claim 33, wherein the pressure space derivative is optimized in the lateral or medial direction.

38. A system comprising:
at least one transducer;
a physiological measurement system; and
at least one processor executing an application, wherein the application causes the at least one processor to at least perform the steps of:
receiving a selection of at least one target anatomy;
computing a plurality of ultrasound parameters for the at least one target anatomy based upon a selected treatment and the at least one target anatomy, wherein one of the ultrasound parameters comprises one or more biological factors determining a frequency or phase of a stimulation generated by the at least one transducer;
generating a placement of the at least one transducer; and
causing the at least one transducer to apply stimulation to the at least one target anatomy.

39. The system of claim 38, wherein the one or more biological factors comprise timing parameters of the brain region or regions being targeted or their connections.

40. The system of claim 38, wherein the one or more biological factors comprise neural activity timing.

41. The system of claim 38, wherein feedback from the physiological measurement system is used to modify the ultrasound waves generated.

42. The system of claim 38, wherein the stimulation comprises ultrasound waves phase-locked to a subject's natural oscillations of the target brain region or regions.

43. The system of claim 38, wherein the stimulation comprises ultrasound waves generated using amplitude modulation of two or more frequencies.

44. The system of claim 38, wherein the stimulation comprises waveforms generated from the gating of two or more waveforms.

45. The system of claim 38, wherein the stimulation comprises waveforms generated from the triggering of two or more waveforms.

46. The system of claim 45, wherein the waveforms are phase offset from one or more frequencies.
